(19) United States
(12) Patent Application Publication
(10) Pub. No.: US 2015/0064252 A1
(43) Pub. Date: Mar. 5, 2015

(54) SOLID DISPERSION FORMULATION OF AN ANTIVIRAL COMPOUND

(71) Applicant: Gilead Pharmasset LLC, Foster City, CA (US)

(72) Inventors: Eric Gorman, San Francisco, CA (US); Erik Mogalian, San Francisco, CA (US); Reza Oliyai, Burlingame, CA (US); Dimitrios Stefanidis, Mountain View, CA (US); Vahid Zia, San Carlos, CA (US)

(21) Appl. No.: 14/168,313

(22) Filed: Jan. 30, 2014

Related U.S. Application Data
(60) Provisional application No. 61/870,703, filed on Aug. 27, 2013.

(51) Int. Cl. A61K 47/32; A61K 31/4188

(52) U.S. Cl. CPC A61K 47/32; A61K 31/4188
USPC 424/482; 424/474; 514/393

(57) ABSTRACT

Disclosed are solid dispersions comprising a compound having the formula:

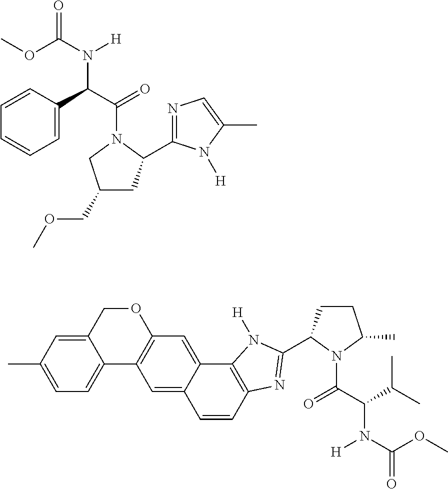

wherein the compound is dispersed within a polymer matrix formed by a pharmaceutically acceptable polymer, and further wherein the compound is substantially amorphous. Also disclosed are pharmaceutical compositions comprising the compound and methods of use for the compound.

### CROSS-REFERENCE TO RELATED APPLICATION

This application claims the benefit under 35 U.S.C. §119(e) to U.S. Provisional Application No. 61/870,703, filed on Aug. 27, 2013, the entirety of which is incorporated herein by reference.

### BACKGROUND

Hepatitis C is recognized as a chronic viral disease of the liver which is characterized by liver disease. Although drugs targeting the liver are in wide use and have shown effectiveness, toxicity and other side effects have limited their usefulness Inhibitors of hepatitis C virus (HCV) are useful to limit the establishment and progression of infection by HCV as well as in diagnostic assays for HCV.

Methyl {(2S)-1-[(2S,5S)-2-(9-{2-[(2S,4S)-1-{(2R)-2-[(methoxycarbonyl)amino]-2-phenylacetyl}-4-(methoxymethyl)pyrrolidin-2-yl]-1H-imidazol-5-yl}-1,11-dihydroisochromeno[4′,3′:6,7]naphtho[1,2-d]imidazol-2-yl)-5-methylpyrrolidin-1-yl]-3-methyl-1-oxobutan-2-yl}carbamate, designated herein as Compound I, is known to be an effective anti-HCV agent (WO 2013/075029 and U.S. Pat. No. 8,575,135).

### SUMMARY

Compound I is a HCV NS5A inhibitor that has demonstrated potent anti-HCV activity against HCV infection of genotypes 1 to 6. Compound I has the following chemical formula:

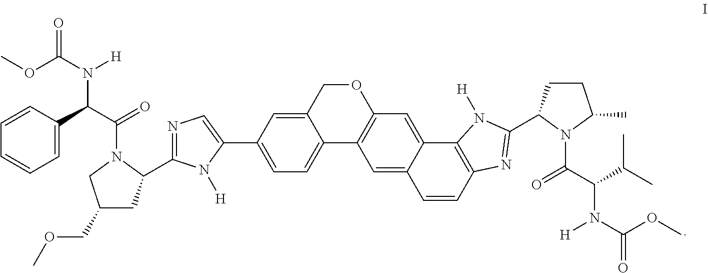 [SMILES: COC[C@H]1C[C@@H](c2ncc(-c3ccc4c(c3)COc3cc5c(ccc6nc([C@@H]7CC[C@H](C)N7C(=O)[C@@H](NC(=O)OC)C(C)C)[nH]c65)cc3-4)[nH]2)N(C(=O)[C@H](NC(=O)OC)c2ccccc2)C1]

Aspects of the disclosure relate to solid dispersions comprising Compound I, wherein Compound I is dispersed within a polymer matrix formed by a pharmaceutically acceptable polymer, and further wherein the Compound I is substantially amorphous.

Further aspects of the disclosure relate to pharmaceutical compositions comprising the solid dispersion and a pharmaceutically acceptable carrier, pharmaceutical dosage forms, and tablets. The disclosure also provides methods for making the solid dispersion and methods for using them in the treatment of hepatitis C.

It is contemplated that the solid dispersions disclosed herein would demonstrate one or more of increased bioavailability, elimination of or reduction in food-effect, reduced negative drug-drug interaction with acid suppressive therapies, reduced variability across patient populations, and/or improved dose linearity at higher doses when compared with administration of Compound I in a conventional formulation.

### BRIEF DESCRIPTION OF THE DRAWINGS

FIG. 1 shows the Fasted State Simulated Intestinal Fluid (FaSSIF) dissolution of amorphous free base and solid dispersion formulations.

FIG. 2 shows the FaSSIF dissolution of acidified solid dispersion formulations (1:1 Compound I “in situ salt”:Copovidone).

### DETAILED DESCRIPTION

### 1. Definitions

As used in the present specification, the following words and phrases are generally intended to have the meanings as set forth below, except to the extent that the context in which they are used indicates otherwise.

As used herein, the term “about” used in the context of quantitative measurements means the indicated amount ±10%. For example, “about 2:8” would mean 1.8-2.2:7.2-8.8.

The term “amorphous” refers to a state in which the material lacks long range order at the molecular level and, depending upon temperature, may exhibit the physical properties of a solid or a liquid. Typically such materials do not give distinctive X-ray diffraction patterns and, while exhibiting the properties of a solid, are more formally described as a liquid. Upon heating, a change from solid to liquid properties occurs which is characterized by a change of state, typically second order (glass transition).

The term “crystalline” refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterized by a phase change, typically first order (melting point).

The term “substantially amorphous” as used herein is intended to mean that greater than 50%; or greater than 55%; or greater than 60%; or greater than 65%; or greater than 70%; or greater than 75%; or greater than 80%; or greater than 85%; or greater than 90%; or greater than 95%, or greater than 99% of the compound present in a composition is in amorphous form. “Substantially amorphous” can also refer to material which has no more than about 20% crystallinity, or no more than about 10% crystallinity, or no more than about 5% crystallinity, or no more than about 2% crystallinity.

The term “polymer matrix” as used herein is defined to mean compositions comprising one or more polymers in which the active agent is dispersed or included within the matrix.

The term “solid dispersion” refers to the dispersion of one or more active agents in a solid state polymer matrix prepared by a variety of methods, including spray drying, the melting (fusion), solvent, or the melting-solvent method.

The term “amorphous solid dispersion” as used herein, refers to stable solid dispersions comprising an amorphous active agent and a polymer. By “amorphous active agent,” it is meant that the amorphous solid dispersion contains active agent in a substantially amorphous solid state form.

The term “pharmaceutically acceptable” indicates that the material does not have properties that would cause a reasonably prudent medical practitioner to avoid administration of the material to a patient, taking into consideration the disease or conditions to be treated and the respective route of administration. For example, it is commonly required that such a material be essentially sterile, e.g., for injectibles.

The term “carrier” refers to a glidant, diluent, adjuvant, excipient, or vehicle with which the compound is administered. Examples of carriers are described herein and also in “Remington's Pharmaceutical Sciences” by E. W. Martin.

The term “polymer” refers to a chemical compound or mixture of compounds consisting of repeating structural units created through a process of polymerization. Suitable polymers useful in this invention are described throughout.

The term “pharmaceutically acceptable polymer” refers to a polymer that does not have properties that would cause a reasonably prudent medical practitioner to avoid administration of the material to a patient, taking into consideration the disease or conditions to be treated and the respective route of administration.

The term “diluent” refers to chemical compounds that are used to dilute the compound of interest prior to delivery. Diluents can also serve to stabilize compounds. Non-limiting examples of diluents include starch, saccharides, disaccharides, sucrose, lactose, polysaccharides, cellulose, cellulose ethers, hydroxypropyl cellulose, sugar alcohols, xylitol, sorbitol, maltitol, microcrystalline cellulose, calcium or sodium carbonate, lactose, lactose monohydrate, dicalcium phosphate, cellulose, compressible sugars, dibasic calcium phosphate dehydrate, mannitol, microcrystalline cellulose, and tribasic calcium phosphate.

The term “binder” when used herein relates to any pharmaceutically acceptable film which can be used to bind together the active and inert components of the carrier together to maintain cohesive and discrete portions. Non-limiting examples of binders include hydroxypropylcellulose, hydroxypropylmethylcellulose, povidone, copovidone, and ethyl cellulose.

The term “disintegrant” refers to a substance which, upon addition to a solid preparation, facilitates its break-up or disintegration after administration and permits the release of an active ingredient as efficiently as possible to allow for its rapid dissolution. Non-limiting examples of disintegrants include maize starch, sodium starch glycolate, croscarmellose sodium, crospovidone, microcrystalline cellulose, modified corn starch, sodium carboxymethyl starch, povidone, pregelatinized starch, and alginic acid.

The term “lubricant” refers to an excipient which is added to a powder blend to prevent the compacted powder mass from sticking to the equipment during the tabletting or encapsulation process. It aids the ejection of the tablet form the dies, and can improve powder flow. Non-limiting examples of lubricants include magnesium stearate, stearic acid, silica, fats, or talc; and solubilizers such as fatty acids including lauric acid, oleic acid, and C₈/C₁₀ fatty acid.

The term “film coating” refers to a thin, uniform, film on the surface of a substrate (e.g. tablet). Film coatings are particularly useful for protecting an active ingredient from photolytic degradation. Non-limiting examples of film coatings include polyvinylalcohol based, hydroxyethylcellulose, hydroxypropylmethylcellulose, sodium carboxymethylcellulose, polyethylene glycol 4000 and cellulose acetate phthalate film coatings.

The term “glidant” as used herein is intended to mean agents used in tablet and capsule formulations to improve flow-properties during tablet compression and to produce an anti-caking effect. Non-limiting examples of glidants include colloidal silicon dioxide, talc, fumed silica, starch, starch derivatives, and bentonite.

The term “therapeutically effective amount” refers to an amount that is sufficient to effect treatment, as defined below, when administered to a mammal in need of such treatment. The therapeutically effective amount will vary depending upon the subject being treated, the weight and age of the subject, the severity of the disease condition, the manner of administration and the like, which can readily be determined by one of ordinary skill in the art.

The term “treatment” or “treating,” to the extent it relates to a disease or condition includes preventing the disease or condition from occurring, inhibiting the disease or condition, eliminating the disease or condition, and/or relieving one or more symptoms of the disease or condition.

The term “% w/w” as used herein refers to the weight of a component based on the total weight of a composition comprising the component. For example, if component A is present in an amount of 50% w/w in a 100 mg composition, component A is present in an amount of 50 mg.

### 2. Solid Dispersions of Compound I

The solid dispersion of Compound I comprises the compound in substantially an amorphous form dispersed within a polymer matrix formed by a pharmaceutically acceptable polymer. The starting material of the solid dispersion can be a variety of forms of Compound I including crystalline forms, amorphous form, salts thereof, solvates and/or the free base. After dispersion with the polymer, the solid dispersion is in the amorphous form.

In one embodiment, the polymer used in the solid dispersion of Compound I is hydrophilic. Non-limiting examples of hydrophilic polymers include polysaccharides, polypeptides, cellulose derivatives such as methyl cellulose, sodium carboxymethylcellulose, hydroxyethylcellulose, ethylcellulose, hydroxypropyl methylcellulose acetate-succinate, hydroxypropyl methylcellulose phthalate, cellulose acetate phthalate, and hydroxypropylcellulose, povidone, copovidone, hypromellose, pyroxylin, polyethylene oxide, polyvinyl alcohol, and methacrylic acid copolymers.

In a further embodiment, the polymer is non-ionic. Non-ionic polymers showed benefits in screening solubility experiments. Non-limiting examples of non-ionic polymers include hypromellose, copovidone, povidone, methyl cellulose, hydroxyethyl cellulose, hydroxypropyl cellulose, ethylcellulose, pyroxylin, polyethylene oxide, polyvinyl alcohol, polyethylene glycol, and polyvinyl caprolactam-polyvinyl acetate-polyethylene glycol (Soluplus®).

In another embodiment, the polymer is ionic. Examples of ionic polymers include hydroxypropyl methylcellulose acetate-succinate, hydroxypropyl methylcellulose phthalate, cellulose acetate phthalate, and methacrylic acid copolymers.

In a further embodiment, the polymer is selected from the group consisting of hypromellose, copovidone, povidone, and polyvinyl caprolactam-polyvinyl acetate-polyethylene glycol (Soluplus®). Copovidone and polyvinyl caprolactam-polyvinyl acetate-polyethylene glycol (Soluplus®) solid dispersions both showed adequate stability and physical characteristics. In a specific embodiment, the polymer is copovidone. In another specific embodiment, the polymer is polyvinyl caprolactam-polyvinyl acetate-polyethylene glycol (Soluplus®).

In certain embodiments, the weight ratio of Compound I to polymer is from about 5:1 to about 1:5. In further embodiments, the weight ratio of Compound I to polymer is about 5:1 to about 1:4, or from about 5:1 to about 1:3, or from about 5:1 to about 1:2, or from about 2:1 to about 1:2, or from about 2:1 to about 1:1. In a specific embodiment, the weight ratio of Compound I to polymer is about 1:1. In another embodiment, the weight ratio of Compound I to polymer is about 1:2. In further embodiments, the weight ratio of Compound I to polymer is about 5:1, 4:1, 3:1, 2:1, 1:1, 1:5, 1:4, 1:3, or 1:2.

### 3. Pharmaceutical Compositions for Oral Delivery

The solid dispersions of Compound I provided in accordance with the present disclosure are usually administered orally. This disclosure therefore provides pharmaceutical compositions that comprise a solid dispersion comprising Compound I as described herein and one or more pharmaceutically acceptable excipients or carriers including but not limited to, inert solid diluents and fillers, diluents, including sterile aqueous solution and various organic solvents, permeation enhancers, solubilizers, disintegrants, lubricants, binders, glidants, adjuvants, and combinations thereof. Such compositions are prepared in a manner well known in the pharmaceutical art (see, e.g., Remington's Pharmaceutical Sciences, Mace Publishing Co., Philadelphia, Pa. 17th Ed. (1985); and Modern Pharmaceutics, Marcel Dekker, Inc. 3rd Ed. (G. S. Banker & C. T. Rhodes, Eds.).

The solid dispersion of Compound I may be present in the pharmaceutical composition in a therapeutically effective amount. In some embodiments, the pharmaceutical composition comprises from about 1% to about 80% w/w of the solid dispersion of Compound I. In further embodiments, the composition comprises from about 1% to about 40% w/w, or from about 2% to about 35% w/w, or from about 3% to about 35% w/w, or from about 3.3% to about 33% w/w, or from about 5% to about 75% w/w, or from about 5% to about 60% w/w, or from about 5% to about 55% w/w, or from about 5% to about 50% w/w, or from about 5% to about 45% w/w, or from about 5% to about 40% w/w, or from about 5% to about 35% w/w, or from about 5% to about 30% w/w, or from about 5% to about 25% w/w, or from about 5% to about 20% w/w, or from about 10% to about 75% w/w, or from about 10% to about 60% w/w, or from about 10% to about 55% w/w, or from about 10% to about 50% w/w, or from about 10% to about 45% w/w, or from about 10% to about 40% w/w, or from about 10% to about 35% w/w, or from about 10% to about 30% w/w, or from about 10% to about 25% w/w, or from about 10% to about 20% w/w, or from about 20 to about 40% w/w of the solid dispersion of Compound I. In a specific embodiment, the pharmaceutical composition comprises about 18% w/w of the solid dispersion of Compound I. In a further specific embodiment, the pharmaceutical composition comprises about 30% of the solid dispersion of Compound I. In further embodiments, the pharmaceutical composition comprises about 3.3% w/w, about 5% w/w, about 10% w/w, about 20% w/w, about 25% w/w, about 30% w/w, about 33% w/w, about 35% w/w, about 40% w/w, or about 45% w/w of the solid dispersion of Compound I. In one embodiment, the pharmaceutical composition comprises about 25% w/w of the solid dispersion of Compound I.

Compound I may be present in the pharmaceutical composition in a therapeutically effective amount. In some embodiments, the pharmaceutical composition comprises from about 1% to about 50% w/w of Compound I. In further embodiments, the composition comprises from about 1% to about 40% w/w, or from about 1% to about 35% w/w, or from about 1% to about 25% w/w, or from about 1% to about 20% w/w, or from about 1% to about 17% w/w, or from about 1% to about 3% w/w, or from about 15% to about 20% w/w of Compound I. In further embodiments, the pharmaceutical composition comprises about 1% w/w, 1.7% w/w, 2% w/w, 5% w/w, about 7% w/w, about 10% w/w, about 12% w/w, about 15% w/w, about 16.7% w/w, about 17% w/w, about 20% w/w, about 25% w/w, about 30% w/w, about 35% w/w, about 40% w/w, about 45% w/w, or about 50% w/w of Compound I. In a specific embodiment, the pharmaceutical composition comprises about 2% w/w of Compound I. In another specific embodiment, the pharmaceutical composition comprises about 17% w/w of Compound I.

In one embodiment, the pharmaceutical composition comprises about 15 to about 30% w/w of a solid dispersion comprising Compound I dispersed within a polymer matrix formed by a pharmaceutically acceptable polymer, wherein the weight ratio of Compound I to polymer is from about 2:1 to about 1:2, or about 1:1.

The pharmaceutical compositions may be administered in either single or multiple doses by oral administration. Administration may be via capsule, tablet or the like. In one embodiment, the compound is in the form of a tablet. In a further embodiment, the tablet is a compressed tablet. In making the pharmaceutical compositions that include the solid dispersion described herein, the solid dispersion is usually diluted by an excipient and/or enclosed within such a carrier that can be in the form of a capsule, tablet, sachet, paper or other container. When the excipient serves as a diluent, it can be in the form of a solid, semi-solid or liquid material (as above), which acts as a vehicle, carrier or medium for the solid dispersion.

The pharmaceutical composition may be formulated for immediate release or sustained release. A “sustained release formulation” is a formulation which is designed to slowly release a therapeutic agent in the body over an extended period of time, whereas an “immediate release formulation” is an formulation which is designed to quickly release a therapeutic agent in the body over a shortened period of time. In some cases the immediate release formulation may be coated such that the therapeutic agent is only released once it reached the desired target in the body (e.g. the stomach). In a specific embodiment, the pharmaceutical composition is formulated for immediate release.

The pharmaceutical composition may further comprise pharmaceutical excipients such as diluents, binders, fillers, glidants, disintegrants, lubricants, solubilizers, and combinations thereof. Some examples of suitable excipients are described herein. When the pharmaceutical composition is formulated into a tablet, the tablet may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

In some embodiments, the pharmaceutical composition comprises lactose monohydrate in an amount from about 0 to about 60% w/w, or from about 10 to about 60% w/w, or from about 20 to about 50% w/w, or from about 30 to about 40% w/w. In specific embodiments, the lactose monohydrate is present at about 20% w/w, at about 25% w/w, at about 30% w/w, at about 35% w/w, or at about 40% w/w. In a further specific embodiment, the lactose monohydrate is in an amount of about 35% w/w.

In further embodiments, the pharmaceutical composition comprises microcrystalline cellulose in an amount from about 10 to about 70% w/w, or from about 20 to about 60% w/w, or from about 25 to about 55% w/w, or from about 20 to about 30% w/w, or from about 50 to about 60% w/w. In specific embodiments, the microcrystalline cellulose is present in an amount of about 10%, or about 15%, or about 20%, or about 25%, or about 26%, or about 30%, or about 35%, or about 40%, or about 45%, or about 50%, or about 55% w/w, or about 56% w/w, or about 60% w/w, or about 65% w/w. In a further specific embodiment, the microcrystalline cellulose is in an amount of about 26% w/w or about 56% w/w.

In further embodiments, the pharmaceutical composition comprises croscarmellose sodium in an amount from about 1 to about 20% w/w, or from about 1 to about 15% w/w, or from about 1 to about 10% w/w, or from about 1 to about 8% w/w, or from about 2 to about 8% w/w. In specific embodiments, the croscarmellose sodium is present in an amount of about 1%, or about 3%, or about 5%, or about 8%, or about 10%, or about 13%, or about 15% w/w. In a further specific embodiment, the croscarmellose sodium is in an amount of about 5% w/w.

In further embodiments, the pharmaceutical composition comprises magnesium stearate in an amount from about 0.1 to about 5% w/w, or from about 0.1 to about 3% w/w, or from about 0.5 to about 3% w/w, or from about 0.5 to about 2.5% w/w, or from about 0.5 to about 2% w/w, or from about 0.5% to about 1.5% w/w, or from about 2 to about 3% w/w. In specific embodiments, the magnesium stearate is present in an amount of about 0.5%, or about 1%, or about 2%, or about 2.5%, or about 3% w/w. In a further specific embodiment, the magnesium stearate is in an amount of about 1% w/w.

In some embodiments, the compositions are formulated in a unit dosage or pharmaceutical dosage form. The term “unit dosage forms” or “pharmaceutical dosage forms” refers to physically discrete units suitable as unitary dosages for human subjects and other mammals, each unit containing a predetermined quantity of active material calculated to produce the desired therapeutic effect, in association with a suitable pharmaceutical excipient (e.g., a tablet or capsule). The compounds are generally administered in a pharmaceutically effective amount. In some embodiments, each dosage unit contains from 3 mg to 2 g of Compound I. In other embodiments, the pharmaceutical dosage form comprises from about 3 to about 500 mg, or about 5 to about 450 mg, or about 5 to about 400 mg, or about 5 to about 350 mg, or about 5 to about 300 mg, or about 5 to about 250 mg, or about 5 to about 200 mg, or about 5 to about 150 mg. In specific embodiments, the pharmaceutical dosage form comprises about 5 mg, or about 10 mg, or about 15 mg, or about 20 mg, or about 25 mg, or about 30 mg, or about 40 mg, or about 50 mg, or about 100 mg, or about 125 mg, or about 150 mg, or about 200 mg, or about 400 mg, or about 450 mg, or about 500 mg, or about 550 mg, or about 600 mg of Compound I. In a further specific embodiment, the pharmaceutical dosage form comprises about 5 mg of Compound I. In a further specific embodiment, the pharmaceutical dosage form comprises about 25 mg of Compound I. In yet a further specific embodiment, the pharmaceutical dosage form comprises about 50 mg of Compound I. In still a further specific embodiment, the pharmaceutical dosage form comprises about 100 mg of Compound I. It will be understood, however, that the amount of the compound actually administered usually will be determined by a physician, in the light of the relevant circumstances, including the condition to be treated, the chosen route of administration, the actual compound administered and its relative activity, the age, weight and response of the individual patient, the severity of the patient's symptoms, and the like.

In one embodiment, the pharmaceutical composition, or alternatively, the pharmaceutical dosage form comprises about 5 mg of Compound I formulated in a solid dispersion comprising a polymer:Compound I ratio of 1:1, and wherein the solid dispersion is in an amount of about 3.3% w/w, lactose monohydrate in an amount from about 25 to about 45% w/w, microcrystalline cellulose in an amount from about 40 to about 65% w/w, croscarmellose sodium in an amount from about 1 to about 10% w/w, and magnesium stearate in an amount from about 0.1 to about 5% w/w. In one embodiment, the polymer is copovidone.

In another embodiment, the pharmaceutical composition, or alternatively, the pharmaceutical dosage form comprises about 25 mg, about 50 mg, or about 100 mg of Compound I formulated in a solid dispersion comprising a polymer:Compound I ratio of 1:1, and wherein the solid dispersion is in an amount of about 33% w/w, lactose monohydrate in an amount from about 25 to about 45% w/w, microcrystalline cellulose in an amount from about 10 to about 40% w/w, croscarmellose sodium in an amount from about 1 to about 10% w/w, and magnesium stearate in an amount from about 0.1 to about 5% w/w. In one embodiment, the polymer is copovidone.

The tablets or pills of the present disclosure may be coated or otherwise compounded to provide a dosage form affording the advantage of prolonged action or to protect from the acid conditions of the stomach. The tablets may also be formulated for immediate release as previously described. In certain embodiments, the tablet comprises a film coating. A film coating of Compound I solid dispersions is useful for limiting photolytic degradation. Suitable film coatings are selected by routine screening of commercially available preparations. In one embodiment, the film coating is a polyvinylalcohol-based coating.

In one embodiment, the tablet comprises a) about 1 to about 40% w/w of the solid dispersion of Compound I; b) about 10 to about 50% w/w lactose monohydrate, c) about 10 to about 60% w/w microcrystalline cellulose, d) about 1 to about 10% w/w croscarmellose sodium, e) about 0.1 to about 10% w/w magnesium stearate, and f) optionally a film coating.

### 4. Methods of Making Solid Dispersions of Compound I

Also provided are methods of making a solid dispersion comprising Compound I. Various techniques are well known in the art for preparing solid dispersions including, but not limited to melt-extrusion, spray-drying, lyophilization, and solution-evaporation.

Melt-extrusion is the process of embedding a compound in a thermoplastic carrier. The mixture is processed at elevated temperatures and pressures, which disperses the compound in the matrix at a molecular level to form a solid solution. Extruded material can be further processed into a variety of dosage forms, including capsules, tablets and transmucosal systems.

For the solution-evaporation method, the solid dispersion can be prepared by dissolving the compound in a suitable liquid solvent and then incorporating the solution directly into the melt of a polymer, which is then evaporated until a clear, solvent free film is left. The film is further dried to constant weight.

For the lyophilization technique, the compound and carrier can be co-dissolved in a common solvent, frozen and sublimed to obtain a lyophilized molecular dispersion.

For spray dried solid dispersions, the solid dispersion can be made by a) mixing the compound and polymer in a solvent to provide a feeder solution; and b) spray drying the feeder solution to provide the solid dispersion.

Spray dried solid dispersions of Compound I provided improved in vivo and in vitro performance and manufacturability/scalability relative to the other formulation approaches, such as wet and dry granulation formulations.

The selection of the polymer for the solid dispersion is based on the stability and physical characteristics of the compound in the solution. Soluplus® and copovidone solid dispersions both showed adequate stability and physical characteristics. Accordingly, in one embodiment, the polymer used in the solid dispersion is selected from Soluplus® and copovidone. Specific embodiments of the invention provide for a 1:2 Compound I:polymer ratio when making the solid dispersion. A further embodiment provides for a 1:1 Compound I:polymer ratio when making the solid dispersion. In one specific embodiment, the polymer used is Soluplus®. In another specific embodiment, the polymer used is copovidone.

After the compound is mixed with the polymer, the mixture can then be solubilized in a solvent. It is within the skill of those in the art to select an appropriate solvent based on the drug and/or polymer properties such as solubility, glass transition temperature, viscosity, and molecular weight. Acceptable solvents include, but are not limited to water, acetone, methyl acetate, ethyl acetate, chlorinated solvents, ethanol, dichloromethane, and methanol. In one embodiment, the solvent is selected from the group consisting of ethanol, dichloromethane, and methanol. In a further embodiment, the solvent is ethanol or methanol. In a specific embodiment, the solvent is ethanol.

Upon solubilization of the compound and polymer mixture with the solvent, the mixture may then be spray dried. Spray drying is a well known process wherein a liquid feedstock is dispersed into droplets into a drying chamber along with a heated process gas stream to aid in solvent removal and to produce a powder product. Suitable spray drying parameters are known in the art, and it is within the knowledge of a skilled artisan in the field to select appropriate parameters for spray drying. The target feed concentration is generally about 10 to about 50% with a target of about 20% and a viscosity of about 1 to 300 cP, or about 1 to 80 cP, or about 4 to 60 cP. The inlet temperature of the spray dry apparatus is typically about 50-190° C., while the outlet temperature is about 30-90° C. The two fluid nozzle and hydraulic pressure nozzle can be used to spray dry Compound I. The two fluid nozzle gas flow can be about 1-100 kg/hr, the hydraulic pressure nozzle flow can be about 15-300 kg/hr, and the chamber gas flow may be about 25-2500 kg/hr. The spray-dried material typically has particle size (D₉₀) less than about 200 μm, or less than about 120 μm, or about 70 to about 80 μm, or in some instances, less than about 25 μm. In some instances, a milling step may be used, if desired to further reduce the particle size. Further descriptions of spray drying methods and other techniques for forming amorphous dispersions are provided in U.S. Pat. No. 6,763,607 and U.S. Patent Publication No. 2006-0189633, the entirety of each of which is incorporated herein by reference.

Spray drying out of ethanol resulted in high yields across a wide range of spray-drying outlet temperatures with no material accumulation on the spray dry chamber. Furthermore, Compound I demonstrated good chemical stability in the ethanolic feed solution.

### 5. Methods of Use

The solid dispersions, pharmaceutical composition, pharmaceutical dosage form, or a tablet of Compound I described herein are administered to a patient suffering from hepatitis C virus (HCV) in a daily dose by oral administration. In one embodiment, the patient is human. In one embodiment, the daily dose is 5 mg, 25 mg, 50 mg, 100 mg, 150 mg, 300 mg, 400 mg, 450 mg, or 600 mg administered in the form of a tablet. In a related embodiment, the tablet comprises a) about 1 to about 40% w/w of the solid dispersion of Compound I; b) about 0 to about 50% w/w lactose monohydrate, c) about 10 to about 60% w/w microcrystalline cellulose, d) about 1 to about 10% w/w croscarmellose sodium, e) about 0.1 to about 10% w/w magnesium stearate, and f) optionally a film coating.

In one embodiment, the solid dispersions, pharmaceutical compositions, pharmaceutical dosage forms, and tablets of Compound I as described herein are effective in treating one or more of genotype 1 HCV infected subjects, genotype 2 HCV infected subjects, genotype 3 HCV infected subjects, genotype 4 HCV infected subjects, genotype 5 HCV infected subjects, and/or genotype 6 HCV infected subjects. In one embodiment, the solid dispersions, pharmaceutical compositions, pharmaceutical dosage forms, and tablets of Compound I as described herein are effective in treating genotype 1 HCV infected subjects, including genotype 1a and/or genotype 1b. In another embodiment, the solid dispersions, pharmaceutical compositions, pharmaceutical dosage forms, and tablets of Compound I as described herein are effective in treating genotype 2 HCV infected subjects, including genotype 2a, genotype 2b, genotype 2c and/or genotype 2d. In another embodiment, the solid dispersions, pharmaceutical compositions, pharmaceutical dosage forms, and tablets of Compound I as described herein are effective in treating genotype 3 HCV infected subjects, including genotype 3a, genotype 3b, genotype 3c, genotype 3d, genotype 3e and/or genotype 3f. In another embodiment, the solid dispersions, pharmaceutical compositions, pharmaceutical dosage forms, and tablets of Compound I as described herein are effective in treating genotype 4 HCV infected subjects, including genotype 4a, genotype 4b, genotype 4c, genotype 4d, genotype 4e, genotype 4f, genotype 4g, genotype 4h, genotype 4i and/or genotype 4j. In another embodiment, the solid dispersions, pharmaceutical compositions, pharmaceutical dosage forms, and tablets of Compound I as described herein are effective in treating genotype 5 HCV infected subjects, including genotype 5a. In another embodiment, the solid dispersions, pharmaceutical compositions, pharmaceutical dosage forms, and tablets of Compound I as described herein are effective in treating genotype 6 HCV infected subjects, including genotype 6a.

In some embodiments, the pharmaceutical composition, pharmaceutical dosage form, or tablet of Compound I as described herein is administered, either alone or in combination with one or more therapeutic agent(s) for treating HCV (such as a HCV NS3 protease inhibitor or an inhibitor of HCV NS5B polymerase), for about 24 weeks, for about 16 weeks, or for about 12 weeks or less. In further embodiments, the pharmaceutical composition, pharmaceutical dosage form, or tablet of Compound I is administered, either alone or in combination with one or more therapeutic agent(s) for treating HCV (such as a HCV NS3 protease inhibitor or an inhibitor of HCV NS5B polymerase), for about 24 weeks or less, about 22 weeks or less, about 20 weeks or less, about 18 weeks or less, about 16 weeks or less, about 12 weeks or less, about 10 weeks or less, about 8 weeks or less, about 6 weeks or less, or about 4 weeks or less. The pharmaceutical composition, pharmaceutical dosage form, or tablet may be administered once daily, twice daily, once every other day, two times a week, three times a week, four times a week, or five times a week.

In further embodiments, a sustained virologic response is achieved at about 24 weeks, at about 20 weeks, at about 16 weeks, at about 12 weeks, at about 10 weeks, at about 8 weeks, at about 6 weeks, or at about 4 weeks, or at about 4 months, or at about 5 months, or at about 6 months, or at about 1 year, or at about 2 years.

### EXAMPLES

In the following examples and throughout this disclosure, abbreviations as used herein have respective meanings as follows:

| | |
|---|---|
| % CV | Percent coefficient of variation |
| AUC | Area Under the Curve |
| AUC_{inf} | Area under the plasma concentration-time |
| | curve from time zero extrapolated to the |
| | infinite time |
| AUCₜₐᵤ | Area under the plasma concentration-time |
| | curve for a dosing interva |
| CL | Drug clearance |
| Cₗₐₛₜ | Last observed plasma concentration |
| Cₘₐₓ | Maximum Concentration |
| cP | Centipoise |
| EC₅₀ | Concentration of a compound inhibiting |
| | birus replication by 50% |
| F | Bioavailability |
| GLSM | Geometric least-squares means |
| h or hr | Hour |
| HCV | Hepatitis C virus |
| ICH | International Conference on Harmonisation; |
| | Impurities guidelines |
| kg | Kilogram |
| m | Meter |
| mg | Milligram |
| mL | Milliliter |
| ng | Nanogram |
| PK | Pharmacokinetics |
| Q1, Q3 | first quartile, third quartile |
| RNA | Ribonucleic Acid |
| s | Second |
| t_{1/2} | Half-life (h) |
| Tₗₐₛₜ | Time of last observed plasma |
| | concentration(h) |
| Tₘₐₓ | Time to reach Cₘₐₓ (h) |
| w | Weight |
| μm | Micrometer |
| DSC | Differential Scanning Calorimetry |
| FaSSIF | Fasted State Simulated Intestinal Fluid |
| USP | United States Pharmacopeia |
| NF | National Formulary |

### Example 1

### Solid Dispersion of Compound I

To make the solid dispersion of Compound I, either a solvate, a salt, or the free base of Compound I can be used.

The spray dry feed solution was prepared by solubilizing Compound I and polymer in the feed solvent. In certain cases, aggressive mixing or homogenization can be used to avoid clumping of the composition.

Different polymers were tested for preferred characteristics in the solid dispersions. Non-ionic such as Soluplus® and copovidone solid dispersions both showed adequate stability and physical characteristics.

The feed solution was initially evaluated for appropriate solvent with regard to solubility, stability, and viscosity. Ethanol, methanol, acetone, and dichloromethane (DCM) all demonstrated excellent solubility. Ethanol and methanol-based feed stocks were assessed for preparation ease and spray dried at a range of inlet and outlet temperatures to assess the robustness of the spray dry process. Both solvents gave rapid dissolution of Compound I and copovidone.

Spray drying out of ethanol resulted in high yields across a wide range of spray-drying outlet temperatures with no material accumulation on the spray dry chamber. Overall, the Compound I solid dispersion in a Compound I to copovidone ratio of 1:1 demonstrated good chemical stability in the ethanolic feed solution.

An ethanolic solution of 10% Compound I and 10% copovidone was prepared using homogenization. Viscosity of ethanolic solutions of Compound I:copovidone were low.

Spray drying was conducted using a commercially available spray dryer (e.g., Anhydro, Buchi, or Niro spray dryer).

Organic volatile impurities, including the spray dry solvent ethanol may be rapidly removed during secondary drying in a tray oven 60° C., purged with room air or via a double cone dryer. Loss on drying can be attributable to water, which can be confirmed by Karl Fischer titration. Residual ethanol was reduced below ICH guidelines of 0.5% w/w by 6 hours of drying.

### Example 2

### Tablet Preparation and Formulation

The following provides an example method for making tablets using the solid dispersions comprising Compound I. The solid dispersion comprising Compound I was blended with excipients and milled to facilitate mixing and blend uniformity.

An in-process milling step may be used to deagglomerate relatively small but hard agglomerates present in the drug substance. To limit any loss of drug substance, Compound I may be blended with all intragranular excipients prior to milling through a conical screen mill, e.g., with a 094R screen and a tip speed of 6 m/s. A secondary blend may be conducted prior to lubrication with magnesium stearate, followed by roller compaction and milling through an in-line oscillating mill. This process results in powder blends with satisfactory flow characteristics and compression properties.

The granules were then mixed with a lubricant prior to tablet compression. The 25 mg, 50 mg, and 100 mg tablets were prepared from a granulation by producing tablet cores of different mass. The 5 mg tablets were prepared from a different granulation containing a lower weight fraction of Compound I solid dispersion, which was offset by increasing the weight fraction of microcrystalline cellulose.

Film-coating of Compound I solid dispersion tablets is provided to reduce photolytic degradation. Tablets were coated to a target 3% weight gain. The film-coating material was a polyvinylalcohol-based coating. Exemplary tablet formulations are provided in Table 1A and Table 1B.

TABLE 1A
Composition of Tablets Comprising the Solid Dispersion of Compound I (5 mg)

| | | | | |
|---|---|---|---|---|
| | | Unit Formula | | |
| | Composition | (mg/tablet) | Quality | |
| Components | (% w/w) | 5 mg | Standard | Function |

Tablet Core

| | | | | |
|---|---|---|---|---|
| Compound I | 1.67 | 5.0 | In-House | Active |
| | | | | Ingredient |
| Copovidone | 1.67 | 5.0 | USP | Polymer |
| Ethanol | N/A | N/A | USP | Processing Aid |
| Lactose Monohydrate | 35.00 | 105.0 | NF | Diluent/Filler |
| Microcrystalline | 55.66 | 167.0 | NF | Diluent/Filler |
| Cellulose | | | | |
| Croscarmellose | 5.00 | 15.0 | NF | Disintegrant |
| Sodium | | | | |
| Magnesium Stearate | 1.00 | 3.0 | NF | Lubricant |
| Total | 100.0 | 300.0 | — | — |

Optional Film Coat

| | | | | |
|---|---|---|---|---|
| Purified Water | N/A | N/A | USP | Processing Aid |
| Opadry II Yellow | 3 | 9.0 | In-House | Film-Coat |

TABLE 1B
Quantitative Composition of Compound I Tablets, 25 mg, 50 mg and 100 mg

| | | Unit Formula | | | | |
|---|---|---|---|---|---|---|
| | Composition | (mg/tablet) | | | Quality | |
| Components | (% w/w) | 25 mg | 50 mg | 100 mg | Standard | Function |

Tablet Core

| | | | | | | |
|---|---|---|---|---|---|---|
| Compound I | 16.67 | 25.0 | 50.0 | 100.0 | In-House | Active |
| | | | | | | Ingredient |
| Copovidone | 16.67 | 25.0 | 50.0 | 100.0 | USP | Polymer |
| Ethanol | N/A | N/A | N/A | N/A | USP | Processing |
| | | | | | | Aid |
| Lactose | 35.00 | 52.5 | 105.0 | 210.0 | NF | Diluent/Filler |
| Monohydrate | | | | | | |
| Microcrystalline | 25.66 | 38.5 | 77.0 | 154.0 | NF | Diluent/Filler |
| Cellulose | | | | | | |
| Croscarmellose | 5.00 | 7.5 | 15.0 | 30.0 | NF | Disintegrant |
| Sodium | | | | | | |
| Magnesium | 1.00 | 1.5 | 3.0 | 6.0 | NF | Lubricant |
| Stearate | | | | | | |
| Total | 100.0 | 150.0 | 300.0 | 600.0 | — | — |

Optional Film Coat

| | | | | | | |
|---|---|---|---|---|---|---|
| Purified Water | N/A | N/A | N/A | N/A | USP | Processing |
| | | | | | | Aid |
| Opadry II | 3 | 4.5 | 9.0 | 18.0 | In-House | Film-Coat |
| Yellow | | | | | | |

### Example 3

### Bioavailability of Compositions Comprising Compound I

Compound I is a novel, potent, specific inhibitor of Hepatitis C Virus Non-Structural 5A (HCV NS5A) protein. To study the pharmacokinetics of Compound I, different formulations were made.

The Compound I formulation is prepared as described above by combining Compound I amorphous free base in a 1:1 ratio by mass with copovidone, which is all dissolved in ethanol and spray dried to produce the Compound I solid dispersion. The Compound I solid dispersion is then mixed with various excipients in a dry granulation process to produce tablets that are film coated prior to packaging.

5 mg, 25 mg, 50 mg, and 100 mg tablet formulations were prepared according to Tables 1A and 1B).

Following single and multiple oral doses of Compound I, maximum plasma concentrations occurred between 1.50 and 3.25 hours (median Tₘₐₓ). Compound I exhibited nonlinear PK across the dose range of 5 to 450 mg. Increases in exposure, as assessed by AUC and Cₘₐₓ, were greater than dose-proportional from 5 to 50 mg and were less than dose-proportional from 50 to 450 mg. Consistent with the half-life of Compound I, modest accumulation was observed with time. After multiple once-daily doses of Compound I greater than 5 mg, the mean plasma concentrations of Compound I at 24 hours postdose were above the protein-adjusted concentration of a compound inhibiting virus replication by 50% (EC₅₀) for genotype 1 to 6 HCV replicons. See Table 2A.

TABLE 2A
Single Dose (Cohorts 1-6^{a})

| | 5 mg | | 50 mg | | 100 mg | | 150 mg | | 450 mg | |
|---|---|---|---|---|---|---|---|---|---|---|
| PK Parameter | (N = 12) | | (N = 12) | | (N = 24) | | (N = 12) | | (N = 12) | |
| AUCₗₐₛₜ | 134.2 | (69.6) | 2970.7 | (40.1) | 4985.6 | (44.8) | 4925.9 | (48.0) | 9503.8 | (34.5) |
| (ng · h/mL) | | | | | | | | | | |
| AUC_{inf} | 158.9 | (64.0) | 3017.2 | (40.1) | 5055.0 | (45.3) | 4978.3 | (47.8) | 9578.1 | (34.3) |
| (ng · h/mL) | | | | | | | | | | |
| Cₘₐₓ (ng/mL) | 22.4 | (55.4) | 371.3 | (32.7) | 574.9 | (37.2) | 608.4 | (46.7) | 1121.6 | (31.7) |
| Cₗₐₛₜ (ng/mL) | 1.40 | (26.9) | 2.34 | (61.4) | 2.85 | (80.3) | 2.23 | (40.1) | 3.28 | (50.5) |

| | | | | | |
|---|---|---|---|---|---|
| Tₘₐₓ (h) | 1.50 | 2.50 | 2.50 | 2.75 | 3.25 |
| | (1.50, 2.00) | (2.00, 3.00) | (2.50, 3.00) | (2.50, 3.50) | (2.50, 3.75) |
| Tₗₐₛₜ (h) | 24.00 | 72.00 | 95.00 | 96.00 | 96.00 |
| | (14.00, 36.00) | (48.00, 96.00) | (71.50, 96.00) | (84.02, 96.00) | (96.00, 96.00) |
| t_{1/2} (h) | 11.20 | 13.62 | 15.73 | 16.16 | 14.97 |
| | (5.40, 16.89) | (10.62, 16.47) | (12.63, 17.11) | (14.55, 17.55) | (12.91, 16.73) |
| CL/F (mL/h) | 58,398.0 | 19,188.4 | 24,617.9 | 72,185.5 | 53,676.4 |
| | (124.4) | (39.2) | (50.8) | (196.4)^{b} | (42.5) |

Multiple Dose (Cohorts 1-4^{a})

| PK Parameter | 5 mg (N = 12) | | 50 mg (N = 12) | | 150 mg (N = 12) | | 450 mg (N = 12) | |
|---|---|---|---|---|---|---|---|---|
| AUCₜₐᵤ | 172.3 | (51.7) | 3032.6 | (40.4) | 4890.8 | (45.4) | 9511.2 | (40.9) |
| (ng · h/mL) | | | | | | | | |
| Cₘₐₓ (ng/mL) | 28.3 | (49.3) | 411.4 | (40.7) | 669.4 | (48.1) | 1195.7 | (38.0) |
| Cₜₐᵤ (ng/mL) | 2.2 | (76.0) | 37.9 | (59.5) | 63.4 | (42.8) | 127.7 | (44.3) |
| Tₘₐₓ (h) | 2.00 | (1.25, 2.50) | 2.50 | (2.25, 3.00) | 2.50 | (2.50, 3.50) | 3.00 | (2.50, 4.25) |
| Tₗₐₛₜ (h) | 24.00 | (24.00, 24.00) | 24.00 | (24.00, 24.00) | 24.00 | (24.00, 24.00) | 24.00 | (24.00, 24.00) |
| t_{1/2} (h) | 13.73 | (13.19, 15.88) | 13.02 | (11.43, 16.23) | 15.15 | (12.03, 15.63) | 11.74 | (10.64, 13.12) |
| CL_{SS}/F (mL/h) | 36,095.7 | (46.4) | 19,593.0 | (50.5) | 45,082.3 | (88.3) | 58,804.6 | (57.3) |

Note:
All PK parameters are reported as mean (% CV), except for Tₘₐₓ, Tₗₐₛₜ, and t_{1/2}, which are reported as median (Q1, Q3).
^{a}Compound I dosing by cohort: Cohort 1 = 50 mg, Cohort 2 = 150 mg, Cohort 3 = 5 mg, Cohort 4 = 450 mg, Cohorts 5 and 6 (pooled in the fasted state) = 100 mg.
^{b}Mean (% CV) CL/F for the Compound I 150 mg group (excluding one patient) was 31,403.8 (40.5) mL/h.

Food slowed the rate of absorption of Compound I, formulated as a solid dispersion, without significantly impacting bioavailability. Administration of Compound I with a light breakfast resulted in 25% and 35% increased AUC and Cₘₐₓ, respectively. Administration of Compound I with a high-fat/high-calorie breakfast resulted in a 14% and 25% decrease in AUC and Cₘₐₓ, respectively. The GLSM ratios and associated 90% CIs (fed/fasted treatments) for AUC were contained within the PK equivalence bounds of 70% to 143% for both meal types. The 90% CIs for Cₘₐₓ were not contained within the 70% to 143% bounds for either meal type. See Table 2B.

TABLE 2B

| | GLSM | | | |
|---|---|---|---|---|
| | Compound I | Compound I | | |
| Compound I PK | 100 mg Fed | 100 mg Fasted | % GLSM Ratio | |
| Parameter | (N = 12) | (N = 12) | (Fed/Fasted) | 90% CI |

Light Breakfast

| | | | | |
|---|---|---|---|---|
| AUCₗₐₛₜ | 6728.66 | 5389.63 | 124.84 | (110.02, 141.67) |
| (ng · h/mL) | | | | |
| AUC_{inf} | 6820.80 | 5469.11 | 124.72 | (109.94, 141.48) |
| (ng · h/mL) | | | | |
| Cₘₐₓ (ng/mL) | 784.70 | 581.72 | 134.89 | (116.84, 155.74) |

High-fat Breakfast

| | | | | |
|---|---|---|---|---|
| AUCₗₐₛₜ | 3222.57 | 3746.30 | 86.02 | (73.17, 101.12) |
| (ng · h/mL) | | | | |
| AUC_{inf} | 3267.75 | 3786.61 | 86.30 | (73.43, 101.42) |
| (ng · h/mL) | | | | |
| Cₘₐₓ (ng/mL) | 364.39 | 485.72 | 75.02 | (62.56, 89.97) |

Note:
Compound I dosing by cohort: Cohort 5 = 100 mg in the fasted and light breakfast states; Cohort 6 = 100 mg in the fasted and high-fat breakfast states.

### Example 4

### Dissolution Studies

Several “in situ salts” were prepared by dissolving the Compound I amorphous free base with copovidone in a mixture of organic solvent and water. Various acidifying agents (sulfuric acid, maleic acid, citric acid, acetic acid, and MSA) were added to generate “in-situ salts” in a spray dried dispersion.

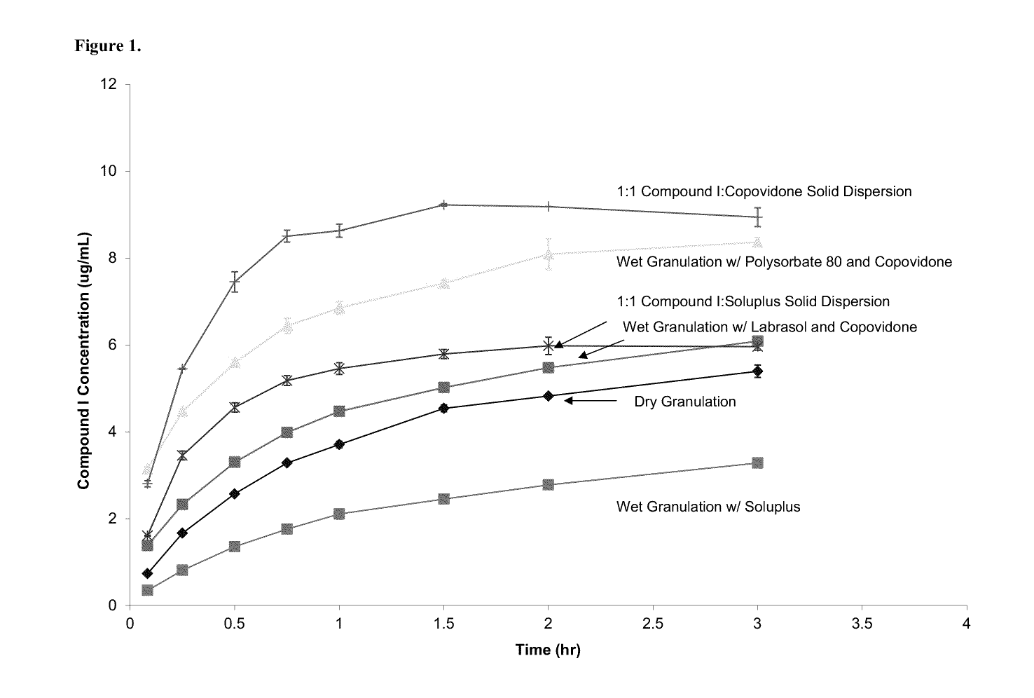

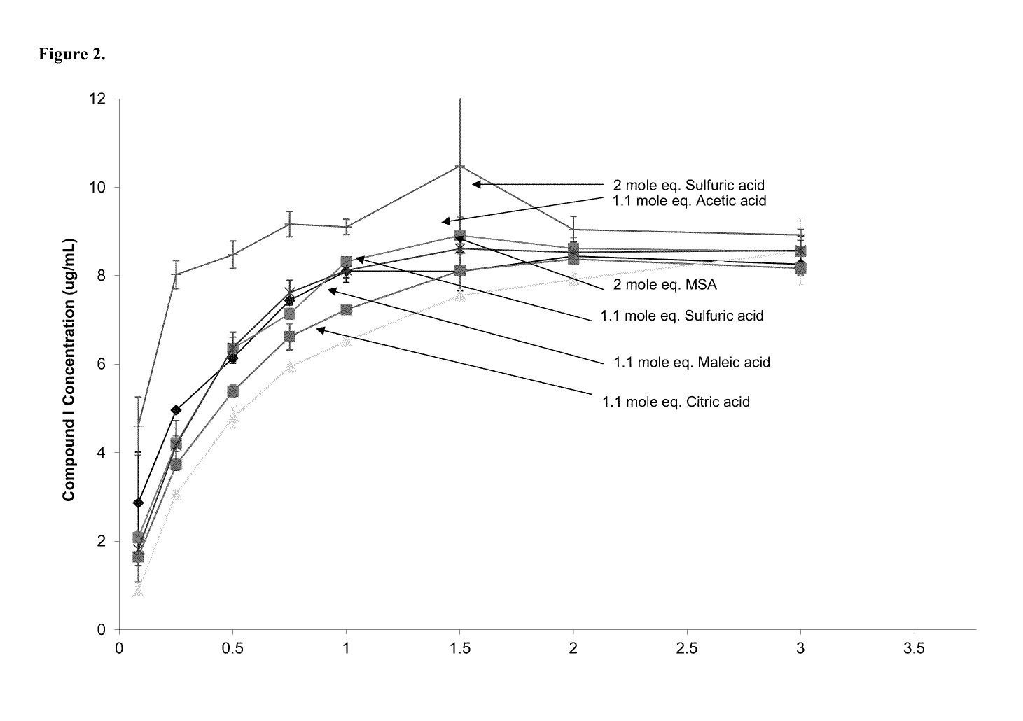

The Dissolution of Compound I tablets in FaSSIF (Fasted State Simulated Intestinal Fluid) was used as an in vitro method to check formulation performance under relatively challenging conditions (i.e. acid suppressed patients) in an effort to minimize food/pH effects in humans (FIG. 1 and FIG. 2). Generally, the results were consistent with the dog PK results (Table 3, Table 4, Table 5, and Table 6).

The wet granulations dissolved as fast or faster than the dry granulation of the amorphous free base formulation. Several other wet granulation formulations were tested (not shown), which typically performed somewhere between the labrasol and polysorbate 80 formulations shown below. The 1:1 Compound I:Copovidone solid dispersion formulation provided the fastest and most complete dissolution of all of the formulations that were tested in these dissolution experiments. Increasing the polymer ratio to 1:2 Compound I:Copovidone did not significantly increase the rate or extent of dissolution in the FaSSIF media (data not shown).

Attemps to generate an “in situ salt” of Compound I in the solid dispersion by the addition of various acidifying agents/counter ions did not significantly improve the rate or extent of dissolution in FaSSIF relative to the original 1:1 Compound I:Copovidone solid dispersion formulation. It was noted that when two molar equivalents of sulfuric acid was added to the solid dispersion the dissolution rate of Compound I did increase. However, this modification only increased the rate of dissolution very slightly with no real impact on the extent of dissolution. Additionally, the presence of the acids may impact the chemical stability of Compound I as under certain storage conditions, physical instability of both of the two molar equivalent materials has been observed. DSC data (not shown) suggests that all acidified solid dispersions are mostly or completely amorphous. In addition, the incorporation of lauric acid in the solid dispersion appears to improve the exposures slightly in dogs.

TABLE 3
25 mg Dose of Compound I in Pentagastrin Treated Dogs

| | | Pentagastrin Treated Dogs | | | |
|---|---|---|---|---|---|
| | | Cₘₐₓ (ng/mL) | | AUC (hr*ng/mL) | |
| | Formulation | Mean | SD | Mean | SD |
| Amorphous | Dry Granulation | 215 | 87 | 1241 | 516 |
| Free | Wet Granulation w/Soluplus | 243 | 52 | 1283 | 287 |
| Base | Wet Granulation w/Labrasol and | 224 | 56 | 1214 | 206 |
| | Copovidone | | | | |
| | Wet Granulation w/Tween 80 and | 250 | 60 | 1423 | 298 |
| | Copovidone | | | | |
| | Wet Granulation w/3% Tween 20 | — | — | — | — |
| | and Copovidone | | | | |
| | Wet Granulation w/5% Tween 20 | — | — | — | — |
| | and Copovidone | | | | |
| | Wet Granulation w/SLS and | 223 | 54 | 1118 | 309 |
| | Copovidone | | | | |
| Solid | 1:1 Compound I:Soluplus | 115 | 39 | 655 | 407 |
| Dispersion | 1:1 Compound I:Copovidone | 206 | 54 | 1183 | 263 |
| | 1:1 Compound I (w/Lauric Acid): | 218 | 33 | 1107 | 191 |
| | Copovidone | | | | |
| | 1:2 Compound I:Copovidone | 239 | 89 | 1533 | 685 |

TABLE 4
25 mg Dose of Compound I in Famotidine Treated Dogs

| | | Famotidine Treated Dogs | | | |
|---|---|---|---|---|---|
| | | Cₘₐₓ (ng/mL) | | AUC (hr*ng/mL) | |
| | Formulation | Mean | SD | Mean | SD |
| Amorphous | Dry Granulation | 23 | 8 | 131 | 57 |
| Free | Wet Granulation w/Soluplus | 14 | 8 | 58 | 34 |
| Base | Wet Granulation w/Labrasol and | 31 | 10 | 135 | 51 |
| | Copovidone | | | | |
| | Wet Granulation w/Tween 80 and | 61 | 9 | 330 | 139 |
| | Copovidone | | | | |
| | Wet Granulation w/3% Tween 20 | 47 | 23 | 258 | 165 |
| | and Copovidone | | | | |
| | Wet Granulation w/5% Tween 20 | 32 | 24 | 146 | 105 |
| | and Copovidone | | | | |
| | Wet Granulation w/SLS and | 44 | 22 | 270 | 129 |
| | Copovidone | | | | |
| Solid | 1:1 Compound I:Soluplus | 38 | 15 | 251 | 79 |
| Dispersion | 1:1 Compound I:Copovidone | 54 | 23 | 258 | 129 |
| | 1:1 Compound I (w/Lauric Acid): | 73 | 38 | 337 | 203 |
| | Copovidone | | | | |
| | 1:2 Compound I:Copovidone | 62 | 26 | 330 | 154 |

TABLE 5
300 mg Dose of Compound I in Pentagastrin Treated Dogs

| | | Pentagastrin Treated Dogs | | | |
|---|---|---|---|---|---|
| | | Cₘₐₓ (ng/mL) | | AUC (hr*ng/mL) | |
| | Formulation | Mean | SD | Mean | SD |
| Amorphous | Dry Granulation | 323 | 125 | 2503 | 1269 |
| Free | Wet Granulation w/Soluplus | 397 | 247 | 3004 | 2210 |
| Base | Wet Granulation w/Labrasol and | 511 | 48 | 3345 | 526 |
| | Copovidone | | | | |
| | Wet Granulation w/Tween 80 and | 539 | 66 | 4245 | 1294 |
| | Copovidone | | | | |
| | Wet Granulation w/3% Tween 20 | — | — | — | — |
| | and Copovidone | | | | |
| | Wet Granulation w/5% Tween 20 | — | — | — | — |
| | and Copovidone | | | | |
| | Wet Granulation w/SLS and | — | — | — | — |
| | Copovidone | | | | |
| Solid | 1:1 Compound I:Soluplus | 329 | 71 | 1958 | 435 |
| Dispersion | 1:1 Compound I:Copovidone | 514 | 263 | 3788 | 1835 |
| | 1:1 Compound I (w/Lauric Acid): | 618 | 240 | 5317 | 2188 |
| | Copovidone | | | | |
| | 1:2 Compound I:Copovidone | 480 | 128 | 3319 | 1262 |

TABLE 6
300 mg Dose of Compound I in Famotidine Treated Dogs

| | | Famotidine Treated Dogs | | | |
|---|---|---|---|---|---|
| | | Cₘₐₓ (ng/mL) | | AUC (hr*ng/mL) | |
| | Formulation | Mean | SD | Mean | SD |
| Amorphous | Dry Granulation | — | — | — | — |
| Free | Wet Granulation w/Soluplus | — | — | — | — |
| Base | Wet Granulation w/Labrasol and | — | — | — | — |
| | Copovidone | | | | |
| | Wet Granulation w/Tween 80 and | 170 | 15 | 1161 | 484 |
| | Copovidone | | | | |
| | Wet Granulation w/3% Tween 20 | — | — | — | — |
| | and Copovidone | | | | |
| | Wet Granulation w/5% Tween 20 | — | — | — | — |
| | and Copovidone | | | | |
| | Wet Granulation w/SLS and | — | — | — | — |
| | Copovidone | | | | |
| Solid | 1:1 Compound I:Soluplus | 254 | 75 | 1739 | 590 |
| Dispersion | 1:1 Compound I:Copovidone | 369 | 95 | 2263 | 833 |
| | 1:1 Compound I (w/Lauric Acid): | — | — | — | — |
| | Copovidone | | | | |
| | 1:2 Compound I:Copovidone | — | — | — | — |

### 3. Conclusions

A solid dispersion of Compound I using spray drying with a hydrophilic polymer was identified to have acceptable stability, physical characteristics, and in vivo performance. A rapidly disintegrating tablet was developed using a dry granulation process and commonly used excipients.

It should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

1. A solid dispersion comprising Compound I, having the formula: 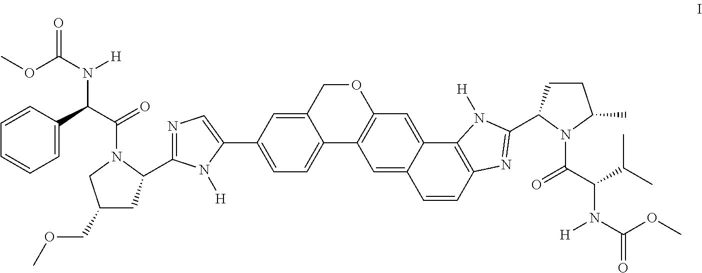 [SMILES: COC[C@H]1C[C@@H](c2ncc(-c3ccc4c(c3)COc3cc5c(ccc6nc([C@@H]7CC[C@H](C)N7C(=O)[C@@H](NC(=O)OC)C(C)C)[nH]c65)cc3-4)[nH]2)N(C(=O)[C@H](NC(=O)OC)c2ccccc2)C1]
wherein the compound is dispersed within a polymer matrix formed by a pharmaceutically acceptable polymer, and further wherein the compound is substantially amorphous.

2. The solid dispersion of claim 1, wherein the polymer is hydrophilic.

3. The solid dispersion of claim 1, wherein the polymer is a non-ionic polymer.

4. The solid dispersion of claim 1, wherein the polymer is selected from the group consisting of hypromellose, hydroxypropyl cellulose, polyvinyl caprolactam-polyvinyl acetate-polyethylene glycol, copovidone, and povidone.

5. The solid dispersion of claim 4, wherein the polymer is copovidone.

6. The solid dispersion of claim 4, wherein the polymer is polyvinyl caprolactam-polyvinyl acetate-polyethylene glycol.

7. The solid dispersion of claim 1, wherein the polymer is an ionic polymer.

8. The solid dispersion of claim 7, wherein the ionic polymer is selected from the group consisting of hydroxypropyl methylcellulose acetate-succinate, hydroxypropyl methylcellulose phthalate, and cellulose acetate phthalate.

9. The solid dispersion of claim 1, wherein the weight ratio of compound to polymer is from about 5:1 to about 1:5.

10. The solid dispersion of claim 9, wherein the weight ratio of compound to polymer is from about 2:1 to about 1:2.

11. The solid dispersion of claim 9, wherein the weight ratio of compound to polymer is about 1:1.

12. The solid dispersion of claim 9, wherein the weight ratio of compound to polymer is about 2:1.

13. A pharmaceutical composition comprising a solid dispersion of claim 1 and a pharmaceutically acceptable carrier.

14. The pharmaceutical composition of claim 9, comprising from about 1% to about 75% w/w of the solid dispersion.

15. The pharmaceutical composition of claim 9, comprising from about 3% to about 35% w/w of the solid dispersion.

16. The pharmaceutical composition of claim 13, wherein the composition is formulated for immediate release.

17. The pharmaceutical composition of claim 13, further comprising one or more of a diluent, a disintegrant, a glidant, a lubricant, and any combination thereof.

18. The pharmaceutical composition of claim 17, wherein the diluent is lactose monohydrate and is present in an amount from about 30 to about 40% w/w.

19. The pharmaceutical composition of claim 17, wherein the disintegrant is microcrystalline cellulose and is present in an amount from about 20 to about 60% w/w.

20. The pharmaceutical composition of claim 17, wherein the disintegrant is croscarmellose sodium and is present in an amount from about 1 to about 10% w/w.

21. The pharmaceutical composition of claim 17, wherein the lubricant is magnesium stearate and is present in an amount from about 0.1 to about 5% w/w.

22. The pharmaceutical composition of claim 13, comprising about 3% w/w of the solid dispersion.

23. The pharmaceutical composition of claim 22, further comprising
a) about 25 to about 45% w/w lactose monohydrate,
b) about 50 to about 60% w/w microcrystalline cellulose,
c) about 1 to about 10% w/w croscarmellose sodium, and
d) about 0.1 to about 5% w/w magnesium stearate.

24. The pharmaceutical composition of claim 13, comprising about 35% w/w of the solid dispersion.

25. The pharmaceutical composition of claim 24, further comprising
a) about 25 to about 45% w/w lactose monohydrate,
b) about 20 to about 30% w/w microcrystalline cellulose,
c) about 1 to about 10% w/w croscarmellose sodium, and
d) about 0.1 to about 5% w/w magnesium stearate.

26. A pharmaceutical dosage form comprising the pharmaceutical composition of claim 13, wherein the dosage form comprises from about 1 to about 300 mg of the compound.

27. The pharmaceutical dosage form of claim 26, wherein the dosage form comprises from about 5 to about 150 mg of the compound.

28. The pharmaceutical dosage form of claim 26, wherein the dosage form comprises about 5 mg, 25 mg, 50 mg, 100 mg or 150 mg of the compound.

29. A tablet comprising the pharmaceutical dosage form of claim 26.

30. The tablet of claim 29, comprising from about 1 to about 150 mg of Compound I.

31. The tablet of claim 29, comprising about 5 mg, 25 mg, 50 mg, 100 mg or 150 mg of Compound I.

32. The tablet of claim 29, further comprising a film coating.

33. The tablet of claim 30, wherein the film coating is a polyvinylalcohol-based coating.

34. The tablet of claim 29, comprising about 3 to about 35% w/w of the solid dispersion.

35. The tablet of claim 34, comprising about 3% w/w of the solid dispersion.

36. The tablet of claim 35 further comprising:
a) about 25 to about 45% w/w lactose monohydrate,
b) about 50 to about 60% w/w microcrystalline cellulose,
c) about 1 to about 10% w/w croscarmellose sodium, and
d) about 0.1 to about 5% w/w magnesium stearate.

37. The tablet of claim 34, comprising about 35% w/w of the solid dispersion.

38. The tablet of claim 37 further comprising:
a) about 25 to about 45% w/w lactose monohydrate,
b) about 20 to about 30% w/w microcrystalline cellulose,
c) about 1 to about 10% w/w croscarmellose sodium, and
d) about 0.1 to about 5% w/w magnesium stearate.

39. A method of treating hepatitis C in a human patient in need thereof comprising administering to the patient a therapeutically effective amount of the solid dispersion of claim 1.

40. A method of making a solid dispersion of claim 1 comprising:
a) mixing Compound I and polymer in a solvent to provide a feeder solution; and
b) spray drying the feeder solution to provide the solid dispersion.

41. The method of claim 40, wherein Compound I is provided as either the free base, salt, or solvate.

42. The method of claim 40, wherein Compound I is provided the free base.

43. The method of claim 40, wherein the solvent is selected from ethanol, methanol, or dichloromethane.

44. The method of claim 40, wherein the solvent is ethanol.
